(19) United States
(12) Patent Application Publication
(10) Pub. No.: US 2002/0197670 A1
(43) Pub. Date: Dec. 26, 2002

(54) Membrane associated progesterone receptor

(76) Inventor: Thomas M. Price, Greenville, SC (US)

Correspondence Address: DORITY & MANNING, ATTORNEYS AT LAW, P.A., Post Office Box 1449, Greenville, SC, 29602, US

(21) Appl. No.: 09/887,280

(22) Filed: Jun. 22, 2001

(51) Int. Cl. C12Q 1/68; C07H 21/04; C12P 21/02; C12N 5/06; C07K 14/72

(52) U.S. Cl. 435/69.1; 435/320.1; 435/325; 435/6; 530/350; 536/23.5

(57) ABSTRACT

The invention provides for human progesterone receptor proteins (PR-M and PR-H) and polynucleotides that identify and encode the proteins. The invention also provides expression factor, host cells, and methods for treating and preventing disorders associated with the expression or binding of the PR-M and/or PR-H receptors. The invention also provides for antibodies, agonists, and antagonists that have utility in the treatment of similar disorders.

### RELATED APPLICATIONS

This application is related to U.S. Application Serial No. 60/213,340, filed Jun. 22, 2000, and which is incorporated herein by reference.

### FIELD OF THE INVENTION

This invention is directed toward nucleic acid and amino acid sequences of novel human progesterone receptor complexes that are membrane bound and found within certain human tissues. The invention relates further to the use of the sequences and useful portions of sequences in the treatment, prevention, and diagnosis of various medical disorders.

### BACKGROUND OF THE INVENTION

Progesterone has been recognized for years as the “pregnancy hormone” of mammals. The ovarian steroid plays a vital role by modulating the cellular processes that are necessary for the development and maintenance of reproductive function. Current studies with transgenic animals have revealed surprisingly that some of the reproductive functions attributed to estrogen are actually mediated by progesterone. Progesterone is now being touted as the “steroid hormone of reproduction”. The effects of progesterone are mediated by unique receptors in reproductive tissues, identified as classical progesterone target tissues. Progesterone receptors (PR) have currently been identified in such nonclassical tissues as bone, prostate, and adipose tissue. The role of progesterone in these tissues has not been elucidated.

PR is a member of the superfamily of intracellular proteins that share structural similarities and function as ligand-activated transcription factors. PR is unique among these steroid receptors by playing a crucial role in the regulation of other steroid hormones. To date, the physiological response to progesterone is known to be conveyed by two discrete forms of PR (PR-A and PR-B, 83 and 116 kDa, respectively) in target tissues. The A receptor is an N-terminally truncated form of the larger PR-B and lacks the first 164 amino acids. Homo and heterodimerization of these isoforms with ligand binding and phosphorylation leads to transcriptional regulation in progesterone-responsive tissues. In general, homodimers of the B isoform act primarily as activators of transcription, whereas PR-A functions as a repressor of PR-B. PR-A can repress transcription not only of PR-B but also of androgen, glucocorticoid, mineralocorticoid, and estrogen receptors.

Several facts suggest that the present information concerning PR may be incomplete and that more than two isoforms exist. Initially, PR appeared to have the narrowest tissue distribution of any steroid receptor, being primarily expressed in the female reproductive tract. PR has recently been detected in such nonclassical tissues as blood vessel walls, prostate stomal cells, and adipose tissue. Just as Estrogen Receptors ER-α and ER-β have been found to be tissue-specific in their distribution, the target tissues for PR may have only just begun to be identified. In addition, PR is unique among the superfamily of steroid receptors in that both isoforms are generated from a single gene, each isoform with its own promoter. Other steroid receptors originate from multiple genes. For example, the two isoforms of ER are derived not only from two different genes, but the genes are located on different chromosomes. Furthermore, the transcriptional regulation of the PR gene appears to be more complicated than that of other steroid receptors in that greater than six transcripts are detected in northern analyses. Northern analyses of ER and glucocorticoid receptor (GR) detect only three transcripts for both forms of each type of receptor. Polyadenylation inadequately explains the number and complexity of PR mRNA's, although the products arise from a single gene. The specificity of the transcripts to PR-A and PR-B has yet to be determined.

Accordingly, as more is learned with respect to PR distribution and functions, there remains room for variation and improvement in the art.

### SUMMARY OF THE INVENTION

The present invention has identified two novel PR proteins in human adipose tissue. The primary structure of at least one of the PR is a membrane protein. It is believed, in accordance with this invention, that adipose tissue contains two novel PR localized in the membrane that function to regulate calcium flux.

Accordingly, it is one aspect of the present invention to provide an isolated and substantially purified polynucleotide sequence encoding the polypeptides comprising the amino acid sequence of SEQ ID NOS: 4, 6, and 8 in which the polypeptide can be used to regulate nongenomic actions of progesterone such as those associated with the regulation of intracellular calcium levels.

It is another aspect of the present inventon to control calcium mobilization within adipocytes and thereby help regulate insulin sensitivity. Since adipose tissue constitutes a major target for insulin resistance, elucidation of a unique PR involved in the control of insulin regulation could potentially impact the prevention and treatment of diabetes. Since insulin resistance is a hallmark for the development of cardiovascular disease, these studies could potentially lead to novel methods for treating and preventing the major cause of mortality in the United States. Moreover, identification of PR isoforms predominant in adipose tissue that regulate adipose tissue physiology affords unique ways to optimize reproductive physiology.

In addition, it is believed that the present invention's novel PR isoforms may be unique to other tissues that have not been considered classical targets for progesterone. Since PR plays such a critical role in overall steroid regulation, the finding of new PRs offers additional further treatment and research options.

It is yet another aspect of the present invention to provide a method for producing a polypeptide comprising the amino acid sequence of SEQ ID NOS: 4 and 6, or one or more fragments thereof, the method comprising the steps of culturing a host cell containing an expression vector having at least a fragment of the polynucleotide sequence encoding PR-H or PR-M; and recovering the polypeptide from the host cell culture.

It is another aspect of the invention to provide a method for obtaining a purified antagonist of the polypeptide of SEQ ID NOS: 4 and 6 or one or more fragments thereof.

Still further, it is another aspect of the present invention to provide a purified agonist of the polypeptide of SEQ ID NOS: 4 and 6 or one or more fragments thereof.

It is yet another aspect of the present invention to provide for a method of treating, diagnosing, or preventing reproductive disorders, immunological disorders, neoplastic disorders, and/or vascular disorders comprising administering to a patient a pharmaceutical composition having an effective amount of an antagonist to PR-H and/or PR-M.

It is yet another aspect of the present invention to provide a method for detecting a polynucleotide which encodes PR-H and/or PR-M in a biological sample comprising the steps of: hybridizing the complement of the polynucleotide sequence that encodes SEQ ID NOS: 1 and 2 to nucleic acid material from a biological sample, thereby forming a hybridization complex; and, detecting the hybridization complex when the presence of the complex correlates with the presence of a polynucleotide encoding PR-H and/or PR-M in the biological sample.

It is yet a further aspect of the present invention to provide for an organ and/or tissue specific agonist or antagonist for the development of specific antibodies that provide improvements for hormone replacement therapy, enhance vasodilatation, and augment beneficial effects of estrogen.

It is yet a further aspect of the present invention to provide for specific agonists or antagonists to PR-H or PR-M that enhance insulin sensitivity and thereby augment treatment options and regimes for insulin deficient patients.

It is yet a further aspect of the present invention to provide for a diagnostic method to identify and visualize tissue and cell types with expressed receptors for PR-M and PR-H.

These and other features, aspects, and advantages of the present invention will become better understood with reference to the following description and appended claims.

### BRIEF DESCRIPTION OF THE DRAWINGS

A full and enabling disclosure of the present invention, including the best mode thereof, to one of ordinary skill in the art, is set forth more particularly in the remainder of the specification, including reference to the accompanying drawings.

FIG. 1 sets forth the nucleic acid sequence set forth in SEQ ID NO: 1 and the identified amino acid sequence of PR-M progesterone receptor, the nucleic acid sequence including upstream and downstream elements adjacent the amino acid encoding sequence.

FIG. 2 sets forth the nucleic acid sequence set forth in SEQ ID NO: 2 and the identified amino acid sequence of PR-H progesterone receptor, the nucleic acid sequence including upstream and downstream elements adjacent the amino acid encoding sequence.

FIG. 3 sets forth the nucleic acid sequence of SEQ ID NO: 3, the sequence being an encoding region of the nucleic acid set forth in SEQ ID NO: 1.

FIG. 4 sets forth the amino acid sequence of SEQ ID NO: 4, the sequence being the expressed protein of the nucleic acid sequence set forth and as seen in SEQ ID NO: 3.

FIG. 5 is the nucleic acid sequence set forth in SEQ ID NO: 5 and is the expressed portion of the sequence set forth in SEQ ID NO: 2.

FIG. 6 is the amino acid sequence set forth in SEQ ID NO: 6 that conforms to the protein expressed by the nucleic acid set forth and as seen in SEQ ID NO: 5.

FIG. 7 is a nucleic acid sequence set forth in SEQ ID NO: 7.

FIG. 8 is the amino acid sequence set forth in SEQ ID NO: 8 which is an expression product of the nucleic acid of SEQ ID NO: 7.

FIG. 9 is a peptide sequence set forth in SEQ ID NO: 9 used in the generation of a polyclonal antibody.

FIG. 10 is the peptide sequence set forth in SEQ ID NO: 10 used in the generation of a polyclonal antibody.

FIG. 11 sets forth a western blot of protein probed with anti-human PR polyclonal antibody.

FIG. 12 sets forth a western analysis using a C19 antibody directed to membrane enriched cell fractions.

FIGS. 13A through 13D set forth fluorescent membrane labeling indicating progesterone having specified binding to the cell membranes.

### DESCRIPTION OF THE INVENTION

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield still a further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

In describing the various figures herein, the same reference numbers are used throughout to describe the same material, apparatus or process pathway. To avoid redundancy, detailed descriptions of much of the apparatus once described in relation to a figure is not repeated in the descriptions of subsequent figures, although such apparatus or process is labeled with the same reference numbers.

As used herein, technical and scientific terms have the same meanings as commonly understood by one of ordinary skill in the relevant art. All publications mentioned herein are incorporated herein by reference for the purpose of describing and disclosing cell lines, vectors, methodologies, or protocols. However, the citation or incorporation of a reference herein is not to be construed that the invention is not entitled to antedate such disclosure by virtue of prior invention.

As used herein, the term “variants” refers to changes in a polynucleotide or protein sequence that provide for substantially identical binding and affinity characteristics of the referenced sequence. In the case of a polynucleotide, a variant or altered sequence would be one that encodes the same or a functionally equivalent protein. Similarly, the encoded protein may be a variant that contains deletions, insertions, or substitutions of amino acid residues that produce a silent change and result in a functionally equivalent protein. Deliberate substitutions may also be made on the basis of similarity in polarity, charge, solubility, and hydrophobicity of the sequences as long as the biological or immunological activity of the PR-H or PAR-M sequences or related fragments are retained.

The physiological response to progesterone is conveyed by two discrete forms of the progesterone receptor (hPR-A and hPR-B) in target tissues. (Wen D, Xu Y, Mais D, al. e. The A and B isoforms of the human progesterone receptor operate through distinct signaling pathways within target cells. Mol Cell Biol. 1994;14:8356-8364) The structure of PRs includes an amino terminus A/B domain that regulates transcription efficiency, the C domain or DNA binding region, the D domain or hinge region responsible for dimerization, and the E/F domain responsible for hormone binding. Homo and heterodimerization of these isoforms with ligand binding and phosphorylation leads to transcriptional regulation in progesterone responsive tissues. The size of these isoforms has been described from human endometrial cells, T47D breast cancer cells and chicken oviduct. In breast cancer cells the B isoform is approximately 116 kDa in size, whereas hPR-A is approximately 83 kDa. Amino acid sequence has been deduced from the cDNA sequenced from T47D cells. The A receptor is an N-terminally truncated form of the larger hPR-B which lacks the first 164 amino acids. These proteins are encoded for by multiple transcripts (11.4 I-IV, 6.1, 5.2, 4.5, 3.2, 2.5 kb), obtained from two promoters, yielding hPR-A and hPR-B specific transcripts. (Gromeyer H, Meyer M, Bocquel M, al. e. Progesterone receptors: isoforms and antihormone activity. J Steroid Bioch Mol Biol. 1991;40:271-278.)

Transcriptional regulation by progesterone is complex and is dependent upon specific activities of the isoforms and the ratio of available isoforms in responsive tissues. In general the B isoform acts primarily as an activator whereas hPR-A functions as a repressor of hPR-B activity. In addition, hPR-A can repress transcriptional activation of glucocorticoid, mineralocorticoid, and estrogen receptors, suggesting that PR-A may play a pivotal role in the regulation of other steroid hormones. Changes in the ratio of A and B isoforms provide another mechanism of regulation of progesterone activity. (Wiehle R, Mangal R, Poindexter A, Weigel N. Human progesterone receptor A and B expression during the menstrual cycle. (Abstract) The Endocrine Society, Washington, D.C. 1995. Graham J, Roman S, McGowan E, Sutherland R, Clarke C. Preferential stimulation of human progesterone receptor B expression by estrogen in T47D human breast cancer cells. J Biol Chem. 1995;270:30693-30700.)

The possibility of additional PRs has been previously suggested. Evidence for this includes the fact that “knockout” experiments showed continued expression of mRNA for PR despite the loss of expression of PR-B and PR-A. Other steroid receptors are known to originate from multiple genes. For example, the two isoforms of Estrogen Receptors (ER) are derived not only from two different genes but the genes are located on different chromosomes. (Enmark E, Pelto-Huikko M, Grandien K, et al. Human ER- gene structure, chromosome localization, and expression pattern. J Clin Endocrinol Metab. 1997;82:4258-4265.)

The transcriptional regulation of the PR gene appears to be more complicated than that of other steroid receptors in that more than six transcripts are detected in northern analyses. (Wei L, Gonzalez-Aller C, Wood W, Miller L, Horwitz K. 5′-Heterogenity in human progesterone receptor transcripts predicts a new amino-terminal truncated “C”-receptor and unique A receptor messages. Mol Endocrinol. 1990;4:1833-1840.) Polyadenylation inadequately explains the number and complexity of PR mRNA's, although the products arise from a single gene.

It has previously been postulated that a third PR receptor exists based on data from numerous studies using a T47D breast cancer cell line. (Wei L, Miner R. Evidence for the existence of a third progesterone receptor protein in human breast cancer cell line T47D. Cancer Res. 1994;54:340-343.) Also, U.S. Pat. No. 5,952,175 sets forth a human PR complex p23-like protein. This patent, is incorporated herein by reference in that the methodologies and protocols set forth herein may be used to advantage with respect to the present sequences of this invention.

Novel PR by Western Analysis in Adipose Tissue and Human Aortic Endothelial Cells (HAEC)

The finding of PRs of different sizes compared to the classic PR-B and A found in uterus and T47D breast cancer cells suggest that there are novel PRs.

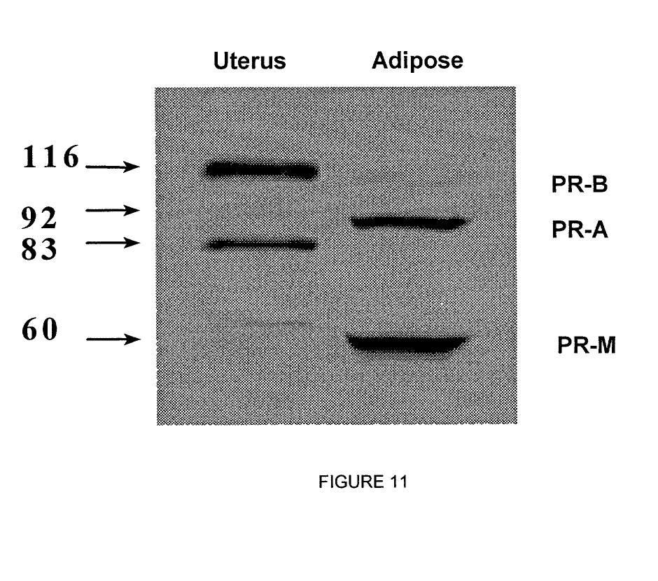

FIG. 11 shows results of a western blot of protein isolated from adipose tissue and uterine myometrium probed with a C19 rabbit anti-human PR polyclonal (Santa Cruz Biotechnology) antibody that is directed to the hormone binding domain, (HBR). The PR-B band is seen at 116 kDa in uterus, T47D (not shown) and adipose. The triplet pattern of PR-B seen in uterus is due to phosphorylation.

PR-A is predominantly seen at 83 kDa in uterus and T47D but at 92 kDa in adipose. The size difference suggests a novel protein. A predominant PR band in adipose tissue is located at approximately 60 kDa. A similar band is seen in T47D cells, of less intensity compared to adipose tissue (not shown). The 60 kDa band is only faintly seen in uterus with overexposure. Controls for this western analysis and the ones shown below include replacement of the primary antibody with a non-specific rabbit IgG, which shows no evidence of nonspecific binding (not shown).

A peptide neutralization assay was then performed to demonstrate that the bands seen at 92 and 60 kDa were specific for the PR.

The C19 primary antibody was pre-incubated with increasing concentrations of antigen to which the antibody was made. While not separately shown, with increasing concentrations of the peptide antigen, the specific bands for the B, A, and M PR isoforms are lost. This suggests that the band at 60 kDa does represent a specific isoform of PR. Further western analyses were then performed with other polyclonal and monoclonal antibodies directed to other regions of the PR. Western analysis using a monoclonal C262 mouse anti-human PR antibody (Santa Cruz Biotechnology), also directed toward the HBR, yielded the same results as with the C19 antibody (not shown).

Additional studies were conducted using western analysis for PR in human aortic endothelial cells (HAEC). This cell type was chosen because of an increasing interest in the control of vascular tone by progesterone and due to the continuing availability of an immortalized cell line as opposed to adipose cells derived from human tissue. Western analysis with the C19 antibody shows a similar pattern to adipose stromal cells with a faint PR-B seen at 116 kDa, the 92 kDa protein discussed above, and the abundant 60 kDa band that is referred to as PR-M.

HAEC Contain a Membrane-Bound PR

After the determination that HAEC contain the same 60 kDa PR band, a determination of whether these cells contain a membrane-bound PR was undertaken.

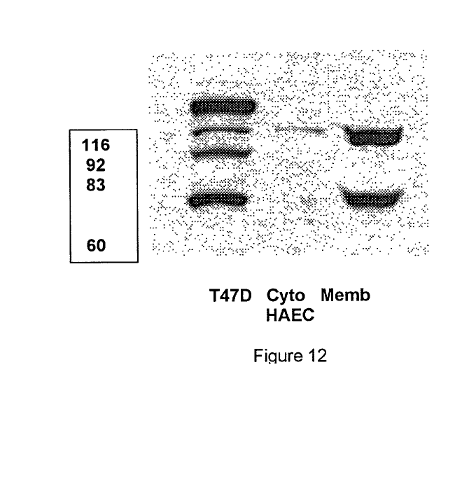

FIG. 12 shows a western analysis with the C19 antibody after differential centrifugation. Differential centrifugation involves separation of cellular membrane from the cytosol fraction by centrifugation at 100,000× g in the presence of protease inhibitors. Although there is some contamination of the cytosol with membrane fragments, the majority of the 60 kDa protein is found in the membrane fraction. Although the majority of the membrane fraction during this technique is composed of plasma membrane, there is also nuclear membrane in the fraction, thus not excluding the possibility that the 60 kDa PR is bound primarily to nuclear membrane. To further investigate the presence of a plasma membrane-bound PR in HAEC, fluorescent membrane labeling studies were conducted.

Fluorescent membrane labeling studies were performed with both ligand (progesterone conjugated to BSA) and with antibody C19 at 4° C. Experimentation at cold temperatures keeps the cells impermeable; thus, ligand or antibody cannot enter the cell, and specific binding is restricted to the membrane.

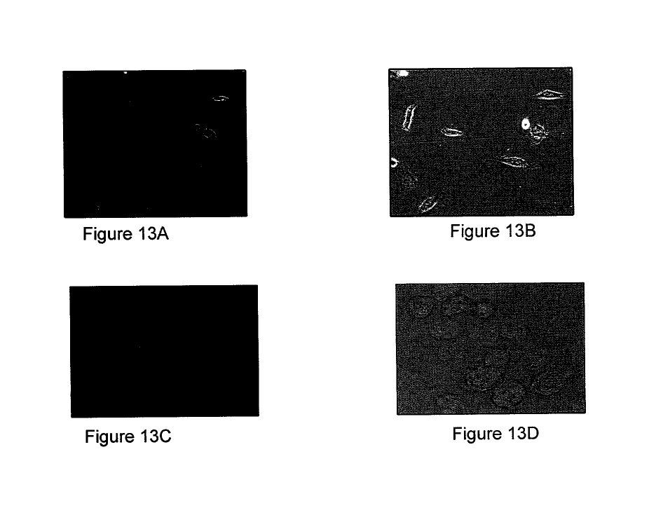

FIG. 13A shows fluorescent membrane labeling with progesterone conjugated to BSA. The staining of the membrane is well seen. In FIG. 13B, a light microscopy image of the same field as in FIG. 13A is shown to illustrate the position of the cells.

Similar results have been found when the C19 antibody is used for fluorescent membrane labeling as seen in the fluorescent staining of FIG. 13C with the corresponding light microscopy in FIG. 13D. Controls used for these experiments include exposure with BSA and the fluorescent tag in the absence of progesterone for the ligand binding study and use of peptide neutralization for the antibody labeling study. All controls show specific membrane binding of progesterone in HAEC. As can be seen in these figures, not all cells stain for the membrane PR. Approximately 10% of all cells show positive staining. This percentage has been found by other investigators in experiments with sperm. The reason for this low percentage of staining is not yet clear. One possibility includes differential expression of the membrane PR during the cell cycle.

Progesterone Treatment of HAEC Causes an Immediate Calcium Response

Aortic endothelial cells were cultured in 100 cm petri dishes. All solutions including washes contained probenecid, a chemical that minimizes leakage of fluorophore from the cells. The medium contained BSA, fluo-3-fluorescence (fluo-3AM), and pluronic F-127, an agent that facilitates diffusion of fluorophore through cell membranes. The cells were incubated for 1 hour at RT. Following incubation, the cells were washed several times and then treated with trypsin. After washing, the cells were enumerated by counting in a hemacytometer and the cell number was adjusted to a predetermined value. Progesterone (50 μM) was added to cell suspensions just prior to fluorescence measurement. An aliquot of the cell suspension was placed in a cuvette and measured in a SLM-8000C fluorescence spectrophotometer. The cells were excited at 504 nm and emitted fluorescence measured at 526 nm.

The data (not shown) establishes an immediate 1.5-fold increase in calcium influx with exposure to progesterone. Since this reaction occurred within 2-3 seconds, it is clear that this is a nongenomic response to a sex steroid.

Identification of Novel PR cDNAs

Protocols were developed to identify novel PR(s) from tissue or cells. This involved the use of RT-PCR to detect differences in transcripts from RNA isolated from adipose tissue and T47D cells (positive control). Using primers selected in regions common to PR-A and PR-B in the sequence below, amplified PCR products were obtained spanning approximately 1100 bp from the 3′ end.

Sequence analyses revealed that PCR product sequences were identical to classical PR in adipose tissue and T47D cells until the junction of exon 1/exon2. Amplification was not successful 5′ of the beginning of exon 2, suggesting that the cDNA was not recognized by upstream primers. This experimentation suggested that there was a novel PR transcript(s) in adipose tissue with unique sequence 5′ of the exon 1/exon 2 junction. From this experimentation, it was not possible to identify specific novel cDNAs. To further define this unique 5′ region, rapid amplification of cDNA ends using adipocyte and Human Aortic cDNA libraries was used.

5′ RACE Using Adipocytes and Human Aortic cDNA Libraries

RACE procedures were performed using Marthon-Ready Adipocyte and Human Aortic cDNA libraries (Clonetech). In this commercially available product, cDNA strands have been modified by addition of specific AP-1 and AP-2 primers onto the 5′ and 3′ ends of the cDNAs. As set forth below, the strategy for the 5′ RACE procedure involved using a 3′ primer in exon 4 at the beginning of the HBR and the 5′ primer corresponding to the AP-1 sequence.

[image]

The 3′ primer was chosen because previous experiments had shown that the C19 antibody directed to the HBR recognized the 60 kDa protein and the RT-PCR described above showed a normal exon 4 sequence present in adipose RNA. Using the 5′ RACE a novel clone was isolated. The sequence of this clone showed exon 4 to be identical to the classical PR. At the exon 3/exon 4 junction there is a sequence encoding 16 novel amino acids in an open reading frame. 5′ to the sequence for these 16 novel amino acids there are 1330 novel base pairs. There are no ATG start sites coding for open reading frames in the 1330 presumed untranslated nucleotides. Protein sequence analysis shows these 16 novel amino acids to be highly hydrophobic followed by a highly hydrophilic region of exon 4. This hydrophobic protein sequence is consistent with a signal peptide, characteristic of a protein either secreted or attached to a membrane.

[image]

3′ RACE Using Adipocytes and Human Aortic cDNA Libraries

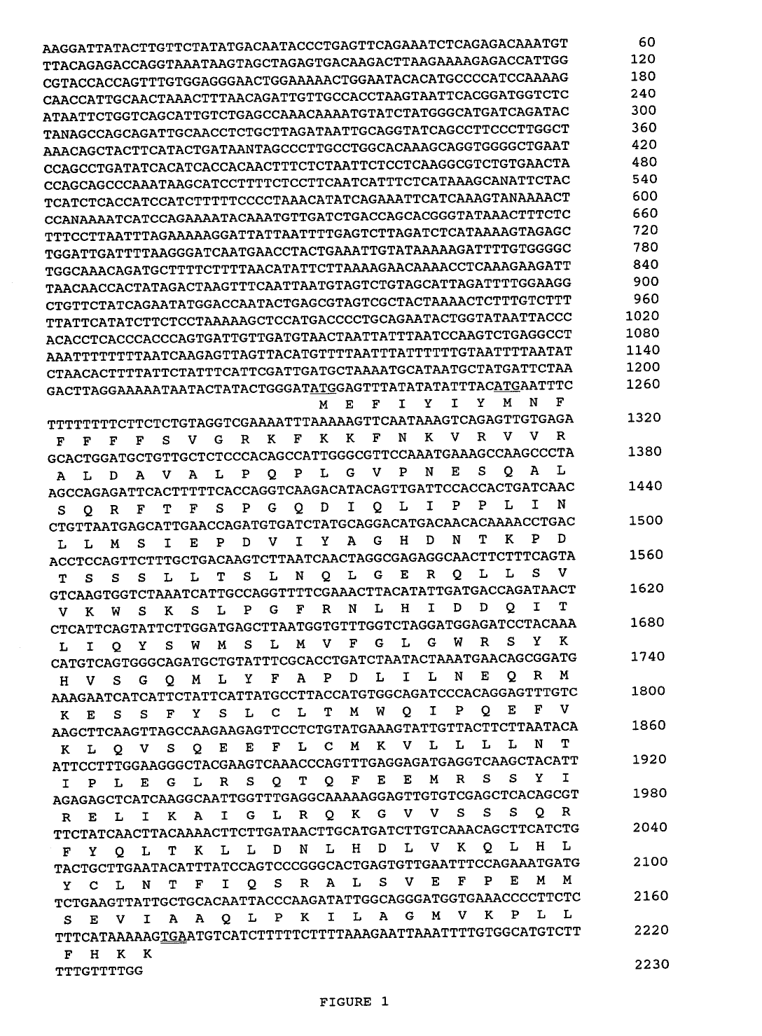

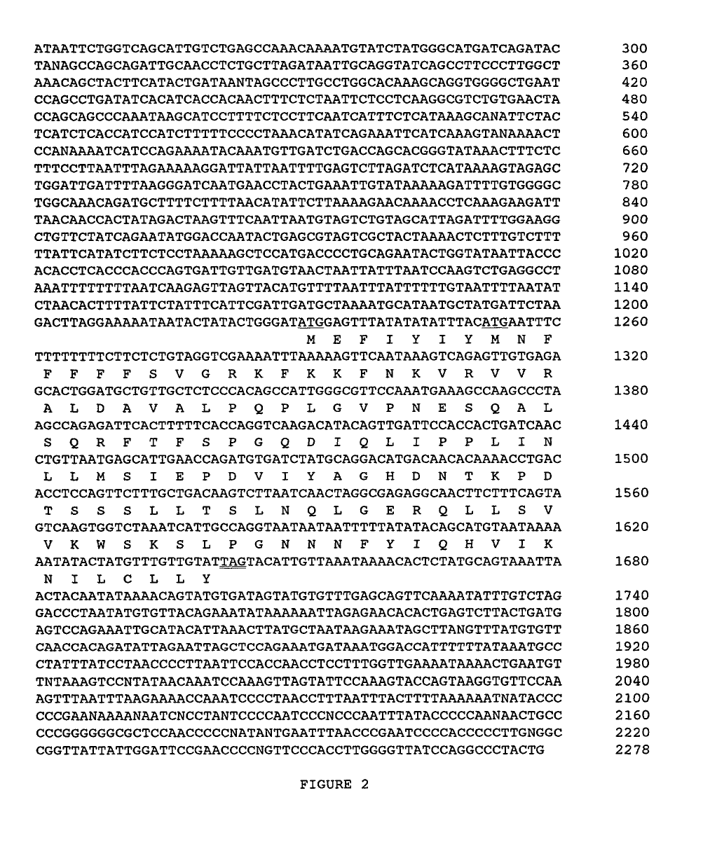

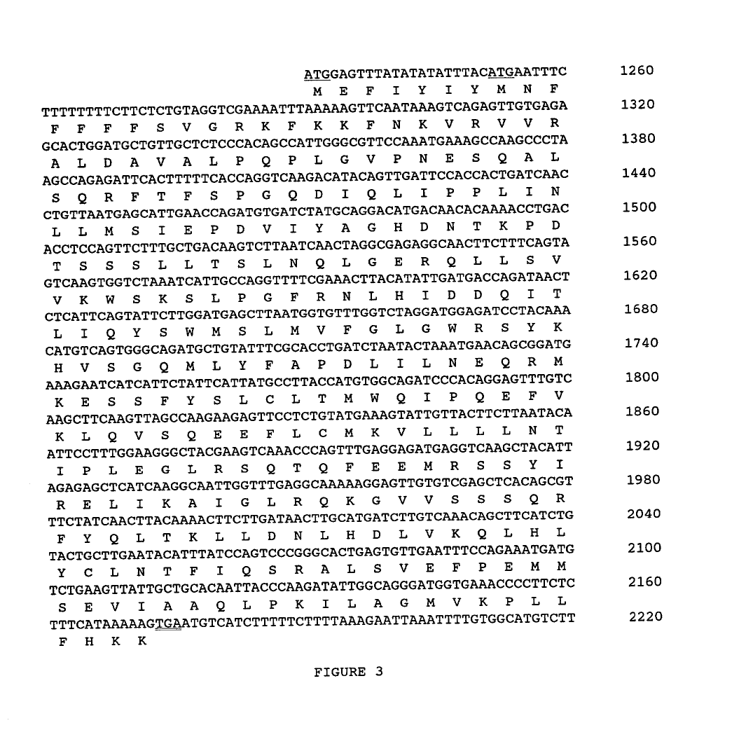

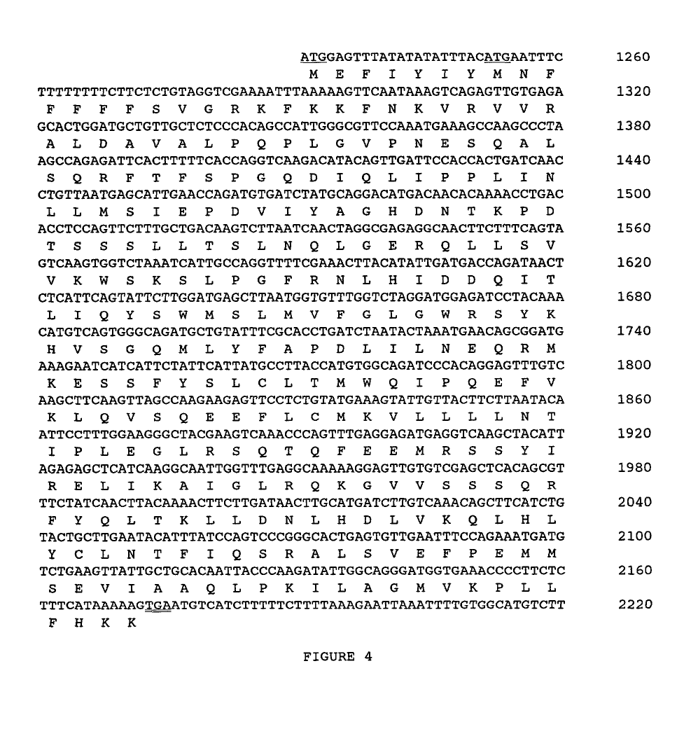

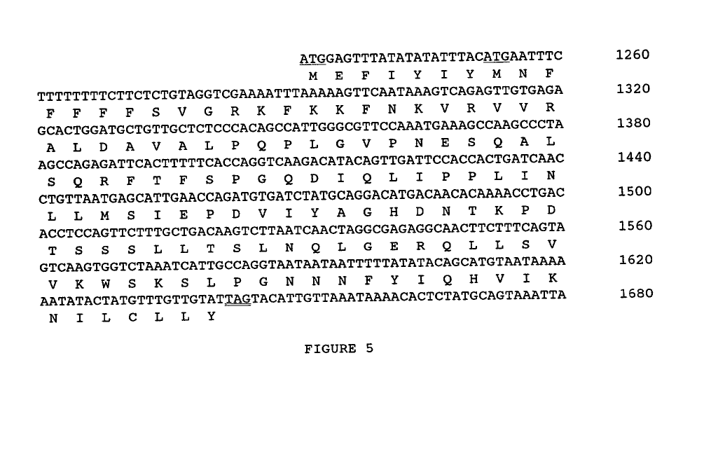

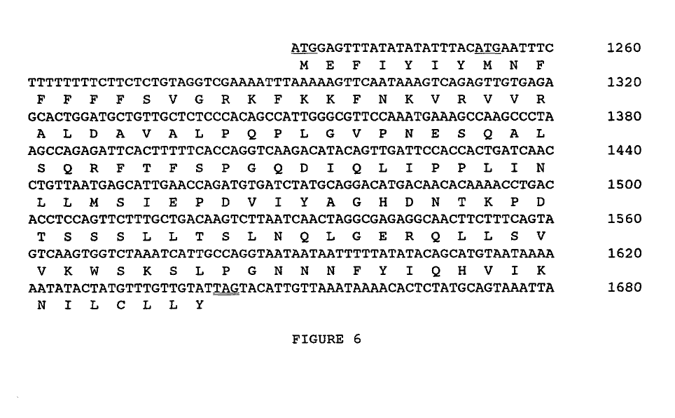

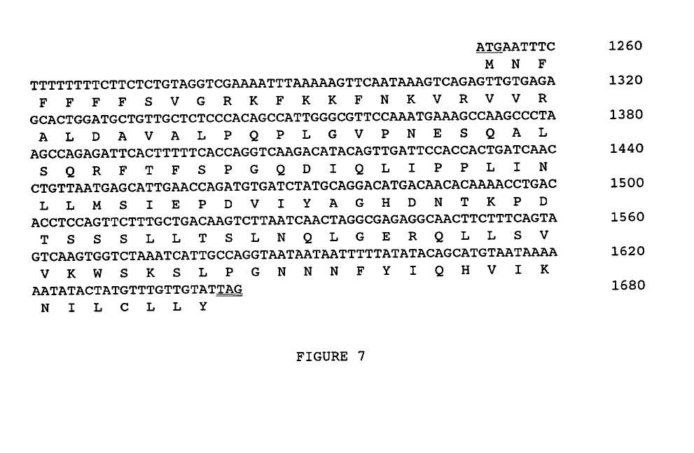

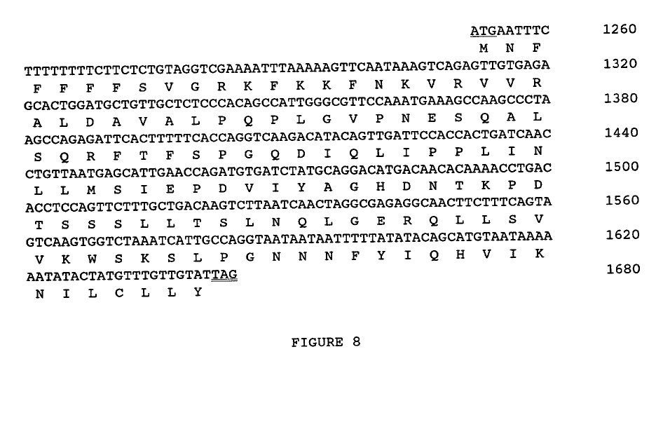

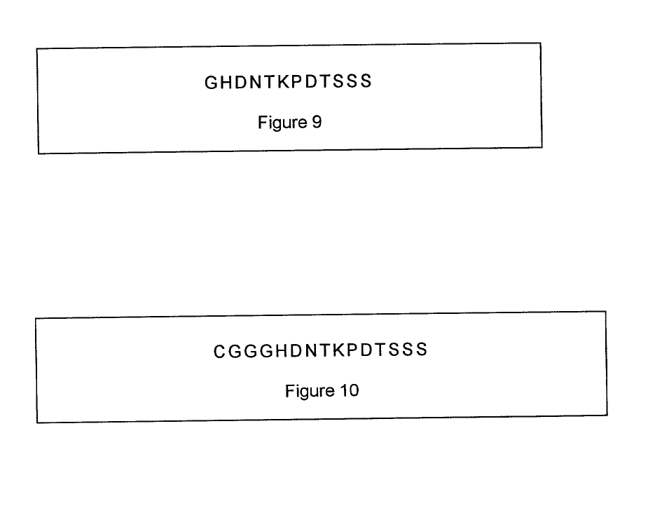

Using the sequence information from the isolated clone described above, 3′ RACE was performed to complete the isolation. The 5′ primer was complimentary to a region of the 1330 novel base pairs while the 3′ primer corresponded to the AP-1 linker at the 3′ end of the cDNA. With this procedure two PR clones were isolated that are designated as PR-hinge and PR-M, as seen in the sequence set forth below and as set forth in FIGS. 1 and 2.

[image]

The sequence for PR-hinge consists of 1330 novel untranslated base pairs. The first in-frame start site is at amino acid 620, which is 16 amino acids before the start of exon 4 found in the classic PR. The sequence for exon 4 in PR-hinge is identical to that of the classic PR. At the end of exon 4, there are 18 novel in-frame amino acids before a stop codon. This stop codon is followed by 64 novel 3′ untranslated base pairs and then a poly-A tail. As discussed above, the novel 16 amino acids at the amino terminus of PR-hinge are characteristic of a signal peptide. The novel 18 amino acids at the carboxy end of the protein are very hydrophobic on a hydrophilicity plot, suggesting a possible interaction with a membrane. The majority of PR-hinge is identical to exon 4 of the classic PR, which encodes for the hinge region (hence the name). This protein is not seen on western analysis, because there are no antibodies, commercial or published, to this region. Likewise, this protein would not have been “knocked-out” in previous mouse experiments (presuming a mouse clone exists).

The sequence for PR-M contains the same 1330 untranslated 5′ base pairs and the same novel 16 amino terminus amino acids. In contrast to PR-hinge, PR-M contains coding sequence identical to exons 4 through 8 of the classic PR. This includes the regions of the hinge and the HBD of the classic PR. This protein is recognized on western analysis by antibodies directed to the HBD such as C19 and C262, but not by an antibody directed to the DNA binding domain such as C20 described above. The estimated size of PR-M is similar to that predicted by Wei and colleagues of approximately 60 kDa. The PR-M protein has an intact hinge region and HBD, and therefore capable of binding ligand and possibly dimerization.

Transcripts Encoding PR-M and PR-hinge Exist in Adipose Tissue and HAEC

After identification of the above clones from human adipocyte and Human Aortic cDNA libraries, the presence of mRNA for PR-M was established in adipose tissue and HAEC by RT-PCR and product sequencing. RT-PCR was performed using total RNA isolated from human adipose tissue obtained from abdominoplasty, and from HAEC grown in culture. First strand synthesis with RT was performed using a 3′ primer in the coding region matching exon 8. PCR was then performed using a 5′ primer in the 1330 bp untranslated region. A PCR product of the correct size was identified and sequenced, matching the sequence data obtained from the cDNA libraries. Similar studies to identify specific transcripts for PR-hinge have been performed. A 3′ primer in the novel 18 amino acid carboxy-end coding region for first strand synthesis, and a 5′ primer in the 1330 bp untranslated region were used. A PCR product of the correct size was identified and then verified by sequencing. To ensure that PR-hinge was not being derived from amplification of contaminating genomic DNA, experiments were performed with DNase treated total RNA and with RT-PCR lacking the RT or primer extension step. RT-PCR experiments with DNase treated total RNA demonstrated the PR-hinge product, which is absent when the reverse transcriptase step is omitted.

Growth and Characterization of HeLa Cells:

HeLa/SF cells from ATCC (CCL-2.3) were used for the transient transfection analyses. These cells are a human cervical adenocarcinoma line that are constitutively PR negative. This SF variant has been adapted to grow in serum-free conditions using TCM, a defined multipurpose serum replacement. Cells are grown in flasks using Dulbecco's modified Eagle's medium with 10 mM HEPES, 98%; TCM, 2%, at 37° C. in 5% CO₂ in T-75 flasks. Phenol red is not used in the medium. HeLa cells were characterized via western blots to ensure that they do not contain protein or transcripts for the classic PR A & B and for the cloned PR-M and PR-hinge.

Total RNA was isolated from HeLa cells using an RNeasy kit (Qiagen, Santa Clarita, Calif.) according to the manufacturer's directions. RT-PCR was performed using the protocol for You-Prime-First-Strand Beads (Pharmacia Biotech). For identification of mRNA for PR-M or PR-hinge, 20 pmol of a reverse primer in the known PR region of exon 4 was used with 2.5 μg total RNA. Subsequent PCR reactions were performed with an oligonucleotide primer in the unique 1330 bp, 5′ untranslated region paired with the reverse primer used in the RT-PCR reaction. An aliquot of this reaction was electrophoresed on a 1% agarose gel to determine size and specificity of amplification.

For sequence analysis, a portion of the PCR product was TA cloned into the pT-Adv vector (Clontech) and sequenced with M13 primers on an ABI 373 automated sequencer. For identification of mRNA encoding PR-A and B, the reverse primer was complimentary to sequence within exon 3 while the 5′ primer matched sequence within exon 1.

Because the HeLa cell line lacks endogenous expression of both the classic PRs and the present invention's cloned PR-M and PR-hinge proteins and transcripts, endogenous production is not a concern. Other similarly dedifferentiated cancer cell types such as COS or CHO cells may show similar utility. Even if a cell line selected expresses PR-M and PR-hinge, useful information and therapeutic protocols may still be developed by overexpression of the proteins and comparison of transfected to non-transfected cells.

Creation of cDNA Vectors

The full length PR-M cDNA and the PR-hinge cDNA, isolated from the Human Aortic cDNA library as described above, are contained in a TA cloning vector (pT-Adv, Clontech). PR-M or PR-hinge cDNA are cut from the TA cloning vector with Eco RI, and purified by agarose gel electrophoresis. Purified PR-M or PR-hinge cDNA are cloned into the Eco RI site of the pCI Mammalian Expression Vector (Promega). Proper cDNA orientation is verified by sequencing on an ABI 373 automated sequencer. The pCI vector has a cytomegalovirus (CMV) promoter for strong constitutive expression.

Transient Transfection

The PR expression vectors are transfected into HeLa/SF cells using a calcium phosphate transfection system (Life Technologies) as set forth in Kilgore M, Tate P, Rai S, Sengoku E, Price T. MCF-7 and T47D human breast cancer cells contain a functional peroxisome response. Molecular and Cellular Endocrinology. 1997;129:229-235. Cells are transfected in 100 mm tissue culture dishes at a density of approximately 10⁶ cells/dish/10 ml medium. Each plate is transfected with 1 ml calcium phosphate—plasmid DNA solution. This process uses a two-tube system. To the first tube is added 1× HBS (HEPES, NaCl), 15 μl NaOH and 10 μl phosphate. To the second tube is added 20 to 40 μg of plasmid DNA followed by 60 μl of calcium solution. The content of the two tubes is then mixed by pipetting and vortexing. The 1 ml mixture is then slowly added to a 100 mm plate of HeLa/SF cells containing 10 ml of growth medium. The plates are incubated for 24 hours, and then fresh media is applied. HeLa/SF cells are transfected with PR-M alone, PR-hinge alone and a combination of PR-M plus PR-hinge.

Determination of PR-M and PR-Hinge Protein Size:

Successful expression of the PR-M transfected protein and determination of protein size was accomplished by conventional western analysis as outlined above. This western analysis included protein from adipose tissue and HAEC cells for a side by side comparison of protein size. Negative controls included protein isolated from non-transfected HeLa/SF cells.

The peptide sequence GHDNTKPDTSSS, derived from the PR-hinge protein, has been used as an antigen for development of a rabbit polyclonal antibody. The process of antigen preparation involves placing a cysteine at the amino terminus with several glycines as spacers so the final peptide sequence is CGGGHDNTKPDTSSS. The cysteine at the amino terminus is conjugated to Keyhole limpet hemocyanin (KLH). KLH is a carrier protein used to create an immunogen for injection. KLH induces a strong antibody response because of its large mass and because it is a non-mammalian protein. The peptide sequence chosen is also ideal because of its hydrophilicity.

Since the hinge region is common to PR-M, PR-hinge, PR-B and PR-A, this antibody may recognize all these proteins. This antibody may also be used in western blot analysis to determine the size of PR-hinge expressed in the transfected cell line.

An alternative antibody recognition protocol may be used in which the transfected protein has added a commercially available V5 epitope (Invitrogen) to the PR protein. The V5 epitope is encoded by a sequence contained in the plasmid immediately after the multiple cloning site. The PR cDNA is constructed without the stop codon so that the final protein contains the V5 epitope at the carboxy terminus. The V5 epitope consists of the amino acid sequence Gly-Lys-Pro-Ile-Pro-Asn-Pro-Leu-Leu-Gly-Leu-Asp-Ser-Thr and adds approximately 3 kb to the size of the protein. The anti-V5 antibody is then used to recognize the PR protein containing the V5 epitope. The epitope affects the size determination of the PR protein, and the epitope may also affect membrane binding or protein-protein interactions of PR protein.

Ligand Membrane-Binding Studies with Transfected HeLa/SF Cells.

HeLa/SF cells transfected with PR-M, PR-hinge or a combination of both PR-M and PR-hinge are grown on a chamber slide system (Lab Tek II) at 37° C. in 5% CO₂. The cells are incubated at 4° C. for 30 minutes with progesterone-conjugated bovine serum albumin-fluorescein isothiocyanate (P-BSA-FITC, Sigma Chemical Co.). At 4° C., the BSA component is unable to enter the cells, thus maintaining the ligand at the membrane. For a negative control, one well is incubated with bovine serum albumin-fluorescein isothiocyanate (BSA-FITC, Sigma Chemical Co.). In addition, non-transfected HeLa/SF cells are analyzed as a negative control, HAEC being used as a positive control. After incubation, the cells are fixed for 2 minutes in freshly prepared 4% paraformaldehyde and mounted for microscopic evaluation with an Olympus 1×70 microscope. Fluorescent signals are collected using Pro Image (Media Cybernetics) software.

Antibody Membrane Binding Studies with Transfected HeLa/SF Cells:

The same protocols outlined above using the ligand are conducted using the C19 antibody and the developed polyclonal antibody. The protocol identifies cell expression for the various cell lines and for indicating binding locations within tissue or cells for both PR-M and PR-hinge in the membrane of transfected cells. PR-M may be identified by the above membrane-binding studies because it contains a ligand-binding domain and is recognized by C19 antibody. PR-hinge should not be identified in these studies because it lacks the ligand-binding domain and the C19 antibody recognition site. PR-hinge will be identified by the developed polyclonal antibody or by the alternate technique for some cell systems. Thus the expression of PR-M may be dependent upon binding to PR-hinge. In this case, only the combined transfection of both PR-M and PR-hinge will demonstrate membrane expression of the protein.

The expressed proteins of PR-M and PR-H and useful peptide fragments thereof may be used for screening of compounds having suitable affinity to the protein of interest. One useful methodology is described in published PCT application WO84/03564, which is incorporated herein by reference. This methodology as applied to PR-H and PR-M peptides uses a large number of different test compounds that are synthesized on a solid substrate. The test compounds are reacted with the PR-H, PR-M, or fragments thereof and washed. Bound PR-H or PR-M may then be detected by methods well known in the art. Purified PR-H and PR-M may also be coated directly onto plates for use in the drug screening techniques referenced above. Alternatively, non-neutralizing antibodies can be used to capture the peptide and immobilize it on a solid support.

One may also use competitive drug screening assays in which neutralizing antibodies that bind PR-H or PR-M specifically compete for binding with a test compound. In this manner, the antibodies can be used to detect the presence of any peptide that shares one or more antigenic determinants with the PR-H, PR-M, or fragments thereof.

Intracellular calcium is measured by monitoring the fluorescence of an indicator (fluorophore) that is loaded into intact cells by incubating them with a membrane permeable ester derivative. Grynkiewicz G, Poenie M, Tsien R. A new generation of Ca 2+ indicators with greatly improved fluorescence properties. J Biol Chem. 1985;260.) Esterases present in the cytosol split off the ester groups and leave the membrane impermeable fluorophore trapped within the cell. Increases in fluorescence correlate to increased calcium ion concentration. Fluorescence of HeLa/SF cell populations is measured by a fluorescent spectrophotometer. Fluorescence of single HeLa/SF cells is measured and quantitated using a fluorescent Olympus 1×70 microscope, and fluorescent signals are collected using a CCD TV camera and image analysis software.

HeLa/SF cells are grown in a chamber slide system and treated with varying concentrations (1 to 50 μM) of progesterone or progestins including medroxyprogesterone acetate and norethindrone acetate. Following treatment, the cells are loaded with fluo-3-fluorescence (fluo-3AM). The cells are excited at 494 nm, and the emitted fluorescence is measured at 526 nm using a SLM-8000C fluorescence spectrophotometer. Intracellular calcium concentration is calculated by the following equation:

[Ca²⁺]=K_{d}×(F−Fₘᵢₙ)/(Fₘₐₓ−F)

K_{d} is the dissociation constant for the fluo-3AM/calcium complex and is calculated from calibration curves using a commercially available kit (Calcium Calibration Kit #1, Molecular Probes). F is the fluorescent signal measured at 526 nm, Fₘᵢₙ is the signal from zero free calcium, and Fₘₐₓ is the signal for saturating free calcium. Each measurement with fluo-3AM-loaded cells is paired with measurements under the same conditions with cells not loaded with fluo-3AM.

Measurement of fluorescence in a single HeLa/SF cell is conducted using a CCD TV camera and Universal Imaging System and a Metafluor Fluorescent Image Acquisition and Processing software. HeLa/SF cells are inoculated at low density on glass cover slips and loaded with fluorophore. Individual isolated cells are identified and measured.

Without being unduly limited by theory, it is believed, in accordance with this invention, that PR-M regulates calcium flux and that HeLa/SF cells transfected with PR-M will have an increase in calcium influx when treated with progesterone. The role of PR-hinge is difficult to predict. It is doubtful that PR-hinge will regulate calcium flux by itself because it lacks a ligand-binding region. It is possible that PR-hinge plays a role in causing a dimerization or clustering of PR-M within the membrane. It is also possible that PR-hinge acts to “anchor” PR-M to the membrane. This could be necessary for the function of PR-M. Nongenomic responses to progesterone are tissue specific. Whereas progesterone increases calcium influx in sperm resulting in an acrosome reaction, progesterone appears to decrease calcium influx in directly treated smooth muscle cells. The nucleic acid and protein sequences directed to the PR-M and PR-H receptors offer new and useful tools to determine the role of progesterone in various cell and tissues types. Antibodies generated to the PR-H and PR-M receptors may include neutralizing antibodies such as those that inhibit dimer formation and may have particular applications for therapeutic use.

That which is claimed is:

1. An isolated and purified polynucleotide sequence encoding the progesterone receptor PR-M of SEQ ID NO:3 and variants thereof.

2. An isolated and purified polynucleotide sequence encoding the progesterone receptor PR-H of SEQ ID NO: 5 and variants thereof.

3. An isolated and purified polynucleotide sequence encoding the progesterone receptor PR-H of SEQ ID NO: 7 and variants thereof.

4. A composition comprising the polynucleotide sequence of the polynucleotide sequence selected from the group consisting of SEQ ID NO: 3, SEQ ID NO: 5, and SEQ ID NO: 7 and combinations thereof.

5. An expression vector containing the polynucleotide sequence of claim 1.

6. An expression vector containing the polynucleotide sequence of claim 2.

7. An expression vector containing the polynucleotide sequence of claim 3.

8. A method for producing a polypeptide comprising an amino acid sequence selected from the group consisting of SEQ ID NO: 4, SEQ ID NO: 6 and SEQ ID NO: 8, the method comprising the steps of:
culturing the host cell containing the polynucleotide sequence selected from the group consisting of SEQ ID NO: 3, SEQ ID NO: 5, and SEQ ID NO: 7 under conditions suitable for the expression of the polypeptide; and,
recovering the polypeptide from the host cell culture.

9. A method for detecting a polynucleotide that encodes a progesterone receptor PR-M protein in a biological sample, the method comprising the steps of:
hybridizing the polynucleotide of claim 1 to nucleic acid material of the biological sample, thereby forming a hybridization complex; and,
detecting the hybridization complex, wherein the presence of the hybridization complex correlates with the presence of a polynucleotide encoding PR-M receptor in the biological sample.

10. An isolated and purified polynucleotide sequence that is complementary to the polynucleotide sequence of claim 4.

11. A method for detecting a polynucleotide that encodes a progesterone receptor PR-M protein in a biological sample, the method comprising the steps of:
hybridizing the polynucleotide of claim 2 to nucleic acid material of the biological sample, thereby forming a hybridization complex; and,
detecting the hybridization complex, wherein the presence of the hybridization complex correlates with the presence of a polynucleotide encoding PR-M receptor in the biological sample.

12. A method for detecting a polynucleotide that encodes a progesterone receptor PR-M protein in a biological sample, the method comprising the steps of:
hybridizing the polynucleotide of claim 3 to nucleic acid material of the biological sample, thereby forming a hybridization complex; and,
detecting the hybridization complex, wherein the presence of the hybridization complex correlates with the presence of a polynucleotide encoding PR-M receptor in the biological sample.
